(19) United States
(12) Patent Application Publication
(10) Pub. No.: US 2021/0213270 A1
(43) Pub. Date: Jul. 15, 2021

(54) RESERVOIR CONNECTOR

(71) Applicant: Biocompatibles UK Limited, Camberley (GB)

(72) Inventors: Maciej Wojciech Misiak, Eden Prairie, MN (US); Andrew Kevin Zachman, St. Michael, MN (US); Ankit Gajurel, Minneapolis, MN (US); Winston Tan, Plymouth, MN (US)

(21) Appl. No.: 17/144,396

(22) Filed: Jan. 8, 2021

Related U.S. Application Data
(60) Provisional application No. 62/959,491, filed on Jan. 10, 2020.

(51) Int. Cl. A61M 39/10; A61M 39/14; A61M 5/14

(52) U.S. Cl. CPC A61M 39/105; A61M 5/1411; A61M 39/14

(57) ABSTRACT

A reservoir connector assembly embodiment includes first and second extensions that are configured to extend into a reservoir. The first extension includes a first extension inner and outer surface and a first extension lumen that is defined by the first extension inner surface. The first extension lumen includes a first aperture that is configured to be positioned within the reservoir. The second extension includes a second extension inner and outer surface and a second extension lumen that is defined by the second extension inner surface. At least a portion of the first extension lumen extends within the second extension lumen. The second extension lumen includes a second aperture that is configured to be positioned within the reservoir. An outlet is in liquid communication with one of the first and second apertures and an inlet is in liquid communication with the other of the first and second apertures.

### CROSS REFERENCE TO RELATED APPLICATION

This application claims priority to Provisional Application No. 62/959,491, filed Jan. 10, 2020, which is herein incorporated by reference in its entirety.

### TECHNICAL FIELD

This disclosure generally relates to the field of medical technology and, more particularly, to devices, systems, and methods for liquid connections in medical technology. For example, embodiments relating to liquid connections for cooling medical energy delivery devices are disclosed herein.

### BACKGROUND

Liquid connections are used in the medical field for a variety of purposes, including facilitating the use of liquid in a number of different medical procedures. For example, in medical procedures involving the delivery of energy, liquid connections can be used to provide liquid as a cooling medium to an energy delivery device. In such an example, the liquid can provide a heat exchange function by drawing heat away from the energy delivery device and thereby helping to keep the energy delivery device operating within desired parameters.

Where liquid is used to cool a medical device, in some cases the liquid is circulated through the medical device to draw off heat and then disposed of. In other cases, the liquid is circulated through the medical device to draw off heat and recirculated through the system to be used again. However, in both cases, a number of liquid connection points are used. In the case of cooling liquid that is disposed of, there is generally an inlet connection at a liquid supply reservoir and another, separate outlet connection at a waste receptacle. In the case of cooling liquid that is recirculated, there is generally one inlet connection at a liquid supply reservoir and another, separate outlet connection at the liquid supply reservoir. These liquid connections can be burdensome to set up, necessitate extra components, and create inefficient liquid flow pathways.

### SUMMARY

In Example 1, a reservoir connector assembly for connecting to a single connection port of a fluid reservoir includes a first extension configured to extend into the single connection port and including a first extension inner surface, a first extension outer surface, and a first extension lumen that is defined by the first extension inner surface, the first extension lumen including a first aperture that is configured to be positioned within the reservoir; a second extension configured to extend into the single connection port and including a second extension inner surface, a second extension outer surface, and a second extension lumen that is defined by the second extension inner surface, at least a portion of the first extension lumen extending within the second extension lumen, the second extension lumen including a second aperture that is configured to be positioned within the reservoir; an outlet in liquid communication with one of the first aperture and the second aperture; and an inlet in liquid communication with the other of the first aperture and the second aperture.

In Example 2, the reservoir connector assembly of Example 1, the second extension outer surface entirely surrounds the first extension lumen.

In Example 3, the reservoir connector assembly of Example 1, a portion of the second extension inner surface and a portion of the first extension outer surface are a common surface.

In Example 4, the reservoir connector assembly of Example 1, the first extension lumen is concentric with the second extension lumen.

In Example 5, the reservoir connector assembly of Example 1, the reservoir connector assembly includes a port connector having an outer surface that is configured to be received in and seal against the single connection port of the reservoir, the outer surface of the port connector being the outer surface of the second extension.

In Example 6, the reservoir connector assembly of Example 1, the first aperture is at a different elevation than the second aperture.

In Example 7, the reservoir connector assembly of Example 1, the first aperture is an output aperture and the outlet is in liquid communication with the first aperture, and the first aperture is at a first end of the first extension lumen and the outlet is at a second end of the first extension lumen, the first end being opposite the second end.

In Example 8, the reservoir connector assembly of Example 1, further including a drip chamber located downstream of the outlet.

In Example 9, the reservoir connector assembly of Example 1, the second aperture is an inlet aperture and the inlet is in liquid communication with the second aperture, and the second aperture is at a first end of the second extension lumen and the inlet is at the second extension inner surface and the second extension outer surface at a second end of the second extension lumen, the first end being opposite the second end.

In Example 10, a reservoir connector assembly for connecting to a connection port of a fluid reservoir includes a first extension configured to extend into the connection port and into the reservoir, the first extension including a first extension inner surface, a first extension outer surface, and a first extension lumen that is defined by the first extension inner surface, the first extension lumen including a first aperture that is configured to be positioned within the reservoir; a second extension configured to extend into the connection port, the second extension including a second extension inner surface, a second extension outer surface, and a second extension lumen that is defined by the second extension inner surface, the second extension lumen including a second aperture that is configured to be positioned within the reservoir, the first extension lumen extending further into the reservoir than the second extension lumen, the first aperture positioned further into the reservoir than the second aperture; an outlet in liquid communication with the second aperture; and an inlet in liquid communication with the first aperture.

In Example 11, the reservoir connector assembly of Example 10, at least a portion of the first extension lumen extends within the second extension lumen.

In Example 12, the reservoir connector assembly of Example 10, a portion of the second extension inner surface and a portion of the first extension outer surface are a common surface.

In Example 13, the reservoir connector assembly of Example 10, the first extension lumen is concentric with the second extension lumen.

In Example 14, the reservoir connector assembly of Example 10, the reservoir connector assembly includes a port connector having an outer surface that is configured to be received in and seal against the connection port of the reservoir, the outer surface of the port connector being the outer surface of the second extension.

In Example 15, the reservoir connector assembly of Example 10, the first extension terminates in a spike for insertion in and penetrating a seal in the connection port, and the second extension terminates in a spike for insertion in and penetrating the seal in the connection port.

In Example 16, the reservoir connector assembly of Example 10, the first aperture is an output aperture and the outlet is in liquid communication with the first aperture, and the first aperture is at a first end of the first extension lumen and the outlet is at a second end of the first extension lumen, the first end being opposite the second end.

In Example 17, the reservoir connector assembly of Example 10, the second aperture is an inlet aperture and the inlet is in liquid communication with the second aperture, and the second aperture is at a first end of the second extension lumen and the inlet is at the second extension inner surface and the second extension outer surface at a second end of the second extension lumen, the first end being opposite the second end.

In Example 18, the reservoir connector assembly of Example 10, the cross-sectional area of the second aperture is larger than the cross-sectional area of the first aperture.

In Example 19, a method of connecting a reservoir connecting assembly to a single connection port of a fluid reservoir, the reservoir connecting assembly includes a first extension including a first extension inner surface, a first extension outer surface, and a first extension lumen that is defined by the first extension inner surface, the first extension lumen including a first aperture that is configured to be positioned within the reservoir; a second extension including a second extension inner surface, a second extension outer surface, and a second extension lumen that is defined by the second extension inner surface, at least a portion of the first extension lumen extending within the second extension lumen, the second extension lumen including a second aperture that is configured to be positioned within the reservoir; connecting the first extension into the single connection port; and connecting the second extension into the single connection port.

In Example 20, the method according to Example 19, further includes connecting a delivery liquid line to an outlet of the reservoir connecting assembly and connecting a return liquid line to an inlet of the reservoir connecting assembly.

In Example 21, a reservoir connector assembly for connecting to a single connection port of a fluid reservoir includes a first extension configured to extend into the single connection port and including a first extension inner surface, a first extension outer surface, and a first extension lumen that is defined by the first extension inner surface, the first extension lumen including a first aperture that is configured to be positioned within the reservoir; and a second extension configured to extend into the single connection port and including a second extension inner surface, a second extension outer surface, and a second extension lumen that is defined by the second extension inner surface, at least a portion of the first extension lumen extending within the second extension lumen, the second extension lumen including a second aperture that is configured to be positioned within the reservoir.

In Example 22, a reservoir connector assembly according to Example 21, the assembly further includes an outlet in liquid communication with one of the first aperture and the second aperture.

In Example 23, a reservoir connector assembly according to Example 21 or 22, the assembly further includes an inlet in liquid communication with the other of the first aperture and the second aperture.

In Example 24, a reservoir connector assembly according to any preceding Example, the second extension outer surface entirely surrounds the first extension lumen.

In Example 25, a reservoir connector assembly according to any preceding Example, a portion of the second extension inner surface and a portion of the first extension outer surface are a common surface.

In Example 26, a reservoir connector assembly according to any preceding Example, the first extension lumen is concentric with the second extension lumen.

In Example 27, a reservoir connector assembly according to any preceding Example, the reservoir connector assembly includes a port connector having an outer surface that is configured to be received in and seal against a single connection port of the reservoir, the outer surface of the port connector being the outer surface of the second extension.

In Example 28, a reservoir connector assembly according to any preceding Example, the first aperture is at a different elevation than the second aperture.

In Example 29, a reservoir connector assembly according to any preceding Example, the first extension and the second extension are each configured to extend past the connection port and into the reservoir.

In Example 30, a reservoir connector assembly according to any preceding Example, the first aperture is an output aperture and the outlet is in liquid communication with the first aperture, and the first aperture is at a first end of the first extension lumen and the outlet is at a second end of the first extension lumen, the first end being opposite the second end.

In Example 31, a reservoir connector assembly according to any preceding Example, the second aperture is an inlet aperture and the inlet is in liquid communication with the second aperture, and the second aperture is at a first end of the second extension lumen and the inlet is at the second extension inner surface and the second extension outer surface at a second end of the second extension lumen, the first end being opposite the second end.

In Example 32, a reservoir connector assembly according to any preceding Example, the first extension terminates in a spike for insertion in and penetrating a seal in the single connection port, and the second extension terminates in a spike for insertion in and penetrating the seal in the single connection port.

In Example 33, a reservoir connector assembly according to any preceding Example, the cross-sectional area of the second aperture is larger than the cross-sectional area of the first aperture.

In Example 34, a reservoir connector assembly according to any preceding Example, the first extension lumen extends further into the reservoir than the second extension lumen, and the first aperture is positioned further into the reservoir than the second aperture.

In Example 35, a reservoir connector assembly according to any preceding Example, further including a drip chamber located downstream of the outlet.

While multiple embodiments are disclosed, still other embodiments will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The following drawings are illustrative of particular embodiments and therefore do not limit the scope of the invention. The drawings are intended for use in conjunction with the explanations in the following description. Embodiments of the invention will hereinafter be described in conjunction with the appended drawings, wherein like numerals denote like elements.

FIG. 1 is a perspective view of an embodiment of a medical system containing a reservoir connector assembly.

FIG. 2 is a perspective view of an embodiment of a reservoir connector assembly of the system shown in FIG. 1.

FIG. 3 is an elevational view of the embodiment of the reservoir connector assembly, shown in FIG. 1, connected to a reservoir.

FIG. 4 is a perspective view of the embodiment of the reservoir connector assembly shown in FIG. 1.

FIGS. 5A and 5B are cross-sectional views, taken along line A-A in FIG. 4, of the embodiment of the reservoir connector assembly shown in FIG. 1. FIG. 5B shows a close-up cross-sectional view of a portion of cross-sectional view of the embodiment of the reservoir connector assembly in FIG. 5A.

FIGS. 6A and 6B are cross-sectional views, taken along a plane perpendicular to line A-A in FIG. 4 at about the height of the extensions shown at line H-H in FIG. 5A, showing different embodiments of the relative positioning of the first extension and the second extension in the embodiment of the reservoir connector assembly shown in FIG. 1.

FIG. 7 is a cross-sectional view showing one embodiment of the relative cross-sectional areas of the extensions in the embodiment of the reservoir connector assembly shown in FIG. 1.

FIGS. 8A-8C are exemplary cross-sectional views of the first extension and the second extension of the reservoir connector assembly.

### DETAILED DESCRIPTION

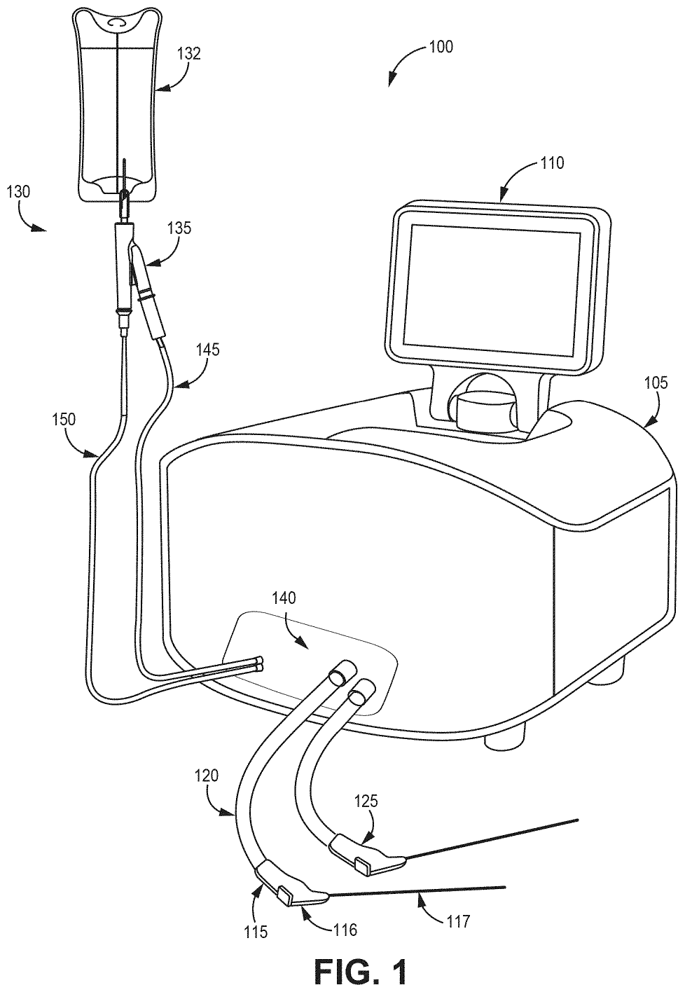

FIG. 1 shows a perspective view of an embodiment of a medical system 100. In the embodiment illustrated here, the medical system 100 is an ablation system. The ablation system can be used in a variety of applications to treat many different conditions. In general, the ablation system can be used to direct energy to a target anatomic region to modify or remove tissue. To do so, energy is passed through the ablation system and used to heat tissue at the target anatomic region. In one example, the ablation system can be an electromagnetic ablation system that directs energy to tissue at the target anatomic region by microwave radiation.

The medical system 100 includes a console 105. The console 105 can include a user interface 110 that can be configured to display information relating to the medical system 100 and receive user input relating to the medical system 100. For example, the user interface 110 can be configured to receive user input relating to operational parameters of the medical system 100, including ablation parameters. The user interface 110 can also be configured to display one or more operational parameters, including ablation parameters, as well as, in some cases, instructions, warnings, and/or error messages relating to the medical system 100 and/or the medical procedure in which the medical system 100 is being used.

The medical system 100 also includes a medical device 115 connected to the console 105. As shown here, the medical device 115 is connected to the console 105 by a line 120. In the illustrated embodiment, the medical device 115 is an electromagnetic ablation device, such as a microwave needle ablation probe. The illustrated embodiment of the medical device 115 includes a handle 116 and a shaft 117. The handle 116 connects to the line 120 and the shaft 117 extends out from the handle 116. The shaft 117 can be configured to be inserted into a patient and to direct energy in the form of microwave radiation to tissue at a target anatomic region. The illustrated embodiment of the medical system 100 is shown as including a second medical device 125 that can be the same as the medical device 115 described herein.

To assist in keeping the medical device 115 operating within desired parameters, the medical system can include a cooling system 130. The cooling system 130 can be configured to provide a heat transfer function at the medical device 115. In particular, the cooling system 130 can be configured to deliver liquid from a reservoir 132 (sometimes referred to as a “liquid reservoir”) through the medical device 115 to draw heat away from the medical device 115. This can help to keep the medical device 115 operating effectively and to prevent the medical device 115 from overheating.

The present disclosure illustrates one embodiment of a cooling system. In the illustrated embodiment, the cooling system 130 includes a reservoir connector assembly 135 and a manifold 140. The reservoir connector assembly 135 can be configured to receive liquid from the reservoir 132 for delivery to the manifold 140 as well as to receive liquid from the manifold 140 for delivery to the reservoir 132. A delivery liquid line 145 and a return liquid line 150 can be included in the cooling system 130 to liquidly connect the reservoir connector assembly 135 and manifold 140. The delivery liquid line 145 can convey liquid from the reservoir connector assembly 135 to the manifold 140, while the return liquid line 150 can convey liquid from the manifold 140 to the reservoir connector assembly 135.

The manifold 140 can be configured to couple to the console 105 and be liquidly connected to the reservoir 132, via the reservoir connector assembly 135, and the medical device 115, via the console 105. The reservoir connector assembly 135 and manifold 140 can thereby facilitate liquid communication between the reservoir 132 and the medical device 115. The medical device 115 and line 120 can include an internal cooling path as part of the cooling system 130 for conveying liquid through the medical device 115 and back to manifold 140. In the illustrated embodiment, the cooling system 130 can be configured as a recirculating cooling system such that liquid is delivered from the reservoir 132 through the reservoir connector assembly 135 and manifold 140 to the medical device 115 and conveyed back from the medical device 115 through the manifold 140 and reservoir connector assembly 135 to the reservoir 132. As such, the medical device 115 can be liquidly connected to the manifold 140 and configured to receive liquid from the delivery liquid line 145 and deliver liquid to the return liquid line 150.

In some further embodiments, the medical system 100 can include one or more other components to help facilitate the flow of liquid through the cooling system 130. For example, in one further embodiment the medical system 100 can include a pump to urge cooling liquid through the medical device 115. The pump could be, for instance, a peristaltic pump that pressurizes liquid to a degree sufficient to circulate through the medical device 115 and back to the reservoir 132. The pump could be located within the console 105 or be an in-line device between the medical device 115 and console 105 or between the reservoir 132 and console 105.

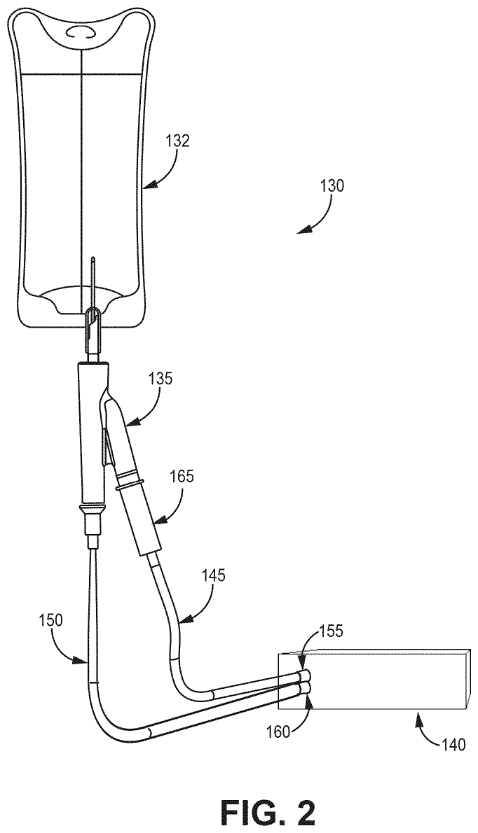

FIG. 2 shows a perspective view of the embodiment of the cooling system 130 used in the medical system of FIG. 1. As noted, the cooling system 130 can include the reservoir connector assembly 135 and the manifold 140. As shown and described in reference to FIG. 1, some further embodiments of the cooling system 130 can include the cooling path within the medical device, the line connecting the medical device to the console, and/or a pump to pressurize liquid circulating within the cooling system 130.

As shown in FIG. 2, the reservoir connector assembly 135 can be liquidly connected with the reservoir 132. Liquid from the reservoir 132 can enter the reservoir connector assembly 135 and flow through the reservoir connector assembly 135 to an outlet of the reservoir connector assembly 135. In one embodiment of the cooling system 130, liquid can pass directly from the outlet of the reservoir connector assembly 135 into the delivery liquid line 145. In an aspect of the cooling system 130, as shown here, a drip chamber 165 can be included and located downstream of the outlet of the reservoir connector assembly 135 and upstream of the delivery liquid line 145. When included, the drip chamber 165 can receive liquid from the outlet of the reservoir connector assembly 135 and can be configured to provide a controlled volume of liquid from the reservoir connector assembly 135 into the delivery liquid line 145.

The manifold 140 can include a manifold inlet 155. The manifold inlet 155 is configured to liquidly connect with the reservoir connector assembly 135 and thereby receive liquid from the reservoir 132. For instance, as shown here, the manifold inlet 155 is liquidly connected to the reservoir connector assembly 135 via the drip chamber 165 and the delivery liquid line 145. In particular, the manifold inlet 155 can be configured to create a liquid tight connection with an end of the delivery liquid line 145.

The manifold 140 can also include one or more device connection ports. Each device connection port can be in liquid communication with the manifold inlet 155 such that liquid received at the manifold inlet 155 is conveyed to the device connection port. Each device connection port can be configured to be in liquid communication with a medical device liquidly connected thereat. As such, each device connection port can convey liquid from the manifold inlet 155 to a cooling path within the medical device. In addition, each device connection port may also be configured to receive liquid circulated through, and output, from the cooling path within the medical device.

The manifold 140 can further include a manifold outlet 160. The manifold outlet 160 can be in liquid communication with each device connection port such that liquid received from the medical device at the device connection port is conveyed to the manifold outlet 160. The manifold outlet 160 is configured to liquidly connect with the reservoir connector assembly 135 and thereby convey liquid to the reservoir 132. For instance, as shown here, the manifold outlet 160 is liquidly connected to the reservoir connector assembly 135 via the return liquid line 150. In particular, the manifold outlet 160 can be configured to create a liquid tight connection with an end of the return liquid line 150.

Liquid from the manifold outlet 160 can enter the reservoir connector assembly 135 at an inlet of the reservoir connector assembly 135. This liquid received at the inlet of the reservoir connector assembly 135 can flow through the reservoir connector assembly 135 and into the reservoir 132. Accordingly, the cooling system 130 can be configured to recirculate liquid between the reservoir 132 and the medical device and, in doing so, carry heat away from the medical device.

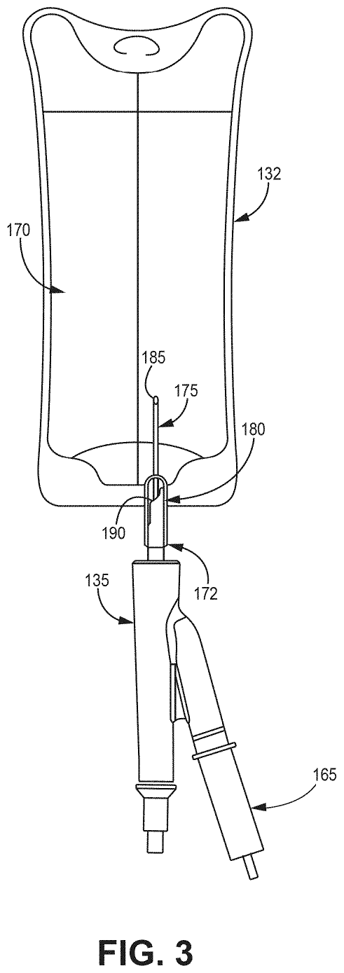

FIG. 3 shows an elevational view of the reservoir connector assembly 135 connected to the reservoir 132. The reservoir 132 can define a main volume 170 within which liquid is contained. Liquid contained within the main volume 170 can be any of a variety of suitable cooling liquid types, such as saline. The reservoir 132 can include a connection port 172 for establishing liquid communication with the main volume 170. More specifically, liquid can be conveyed out of the main volume 170, and delivered into the main volume 170, via the connection port 172. Connection port 172 may be of the type commonly used for one-way saline delivery to a patient. As described further below, though, connection port 172 may be used for two-way liquid flow, including both delivery from the reservoir 132 to the manifold 140 and return from the manifold 140 back to the reservoir 132.

As shown in FIG. 3, the reservoir connector assembly 135 can be in liquid connection with the main volume 170 of the reservoir 132 via the connection port 172. The reservoir connector assembly 135 can include a first extension 175 configured to extend into the reservoir 132 and a second extension 180 configured to extend into the reservoir 132. One or both of the first extension 175 and the second extension 180 can include a pointed tip that may help to pierce through the connection port 172 (forming a single aperture) as the first and second extensions 175, 180 are placed in liquid communication with the main volume 170. In the illustrated embodiment, the first extension 175 and the second extension 180 are each configured to extend past the connection port 172 of the reservoir 132 and into the main volume 170 of the reservoir 132. For instance, as shown here, a first aperture 185 at the first extension 175 is configured to be positioned within the reservoir 132 and a second aperture 190 at the second extension 180 is configured to be positioned within the reservoir 132. In such an embodiment, each of the first aperture 185 and the second aperture 190 can have independent liquid communication with the main volume 170 of the reservoir 132. Such a configuration can allow for liquid to be drawn from the main volume 170 at one of the first aperture 185 and second aperture 190 and returned to the main volume 170 from the other of the first aperture 185 and the second aperture 190 via the one connection port 172 to the reservoir 132.

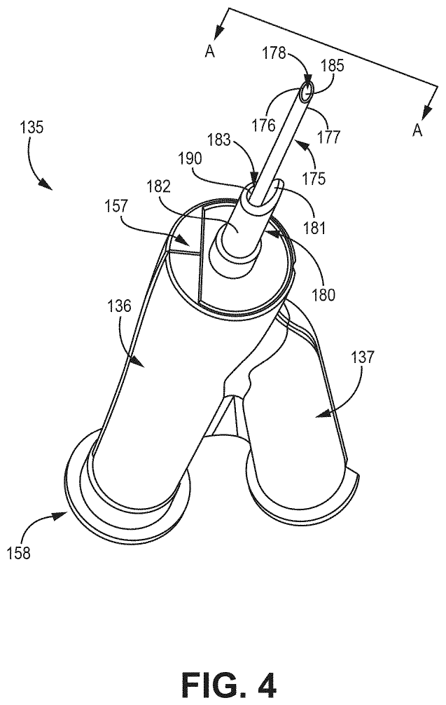

FIG. 4 shows a perspective view of the reservoir connector assembly 135. As noted, the reservoir connector assembly 135 includes the first extension 175 and the second extension 180. In the illustrated embodiment, each of the first extension 175 and the second extension 180 can extend out from a main body 136 of the reservoir connector assembly 135. For example, each of the first extension 175 and the second extension 180 can extend out from a main body 136 at an end 157 of the main body 136 opposite another end 158 on the main body 136.

The first extension 175 includes a first extension inner surface 176, a first extension outer surface 177, and a first extension lumen 178. The first extension lumen 178 can be defined by the first extension inner surface 176. The first extension lumen 178 includes the first aperture 185 at an end of the first extension 175 that is configured to be positioned within the reservoir 132. As such, the first extension lumen 178 can be configured to be in liquid communication with the reservoir 132 at the first aperture 185.

The second extension 180 includes a second extension inner surface 181, a second extension outer surface 182, and a second extension lumen 183. The second extension lumen 183 can be defined by the second extension inner surface 181. The second extension lumen 183 includes the second aperture 190 at an end of the second extension lumen 183 that is configured to be positioned within the reservoir 132. As such, the second extension lumen 183 can be configured to be in liquid communication with the reservoir 132 at the second aperture 190.

In the illustrated embodiment, a portion of the first extension 175 extends within the second extension 180. Accordingly, in the illustrated embodiment, at least a portion of the first extension lumen 178 extends within the second extension lumen 183. In this case, the second extension lumen 183 circumvents the portion of the first extension lumen 178 extending within the second extension lumen 183. As shown here, the first extension 175 is received at, and extends through, the second aperture 190 of the second extension 180. In order to accommodate this configuration, a cross-sectional area of the second extension lumen 183 is greater than a cross-sectional area of the first extension lumen 178. More specifically, in this case, an inner width defined between opposite ends of the second extension inner surface 181 is greater than an outer width defined between opposite ends of the first extension outer surface 177. In some further embodiments, a cross-sectional area of the second aperture 190 can be greater than a cross-sectional area of the first aperture 185.

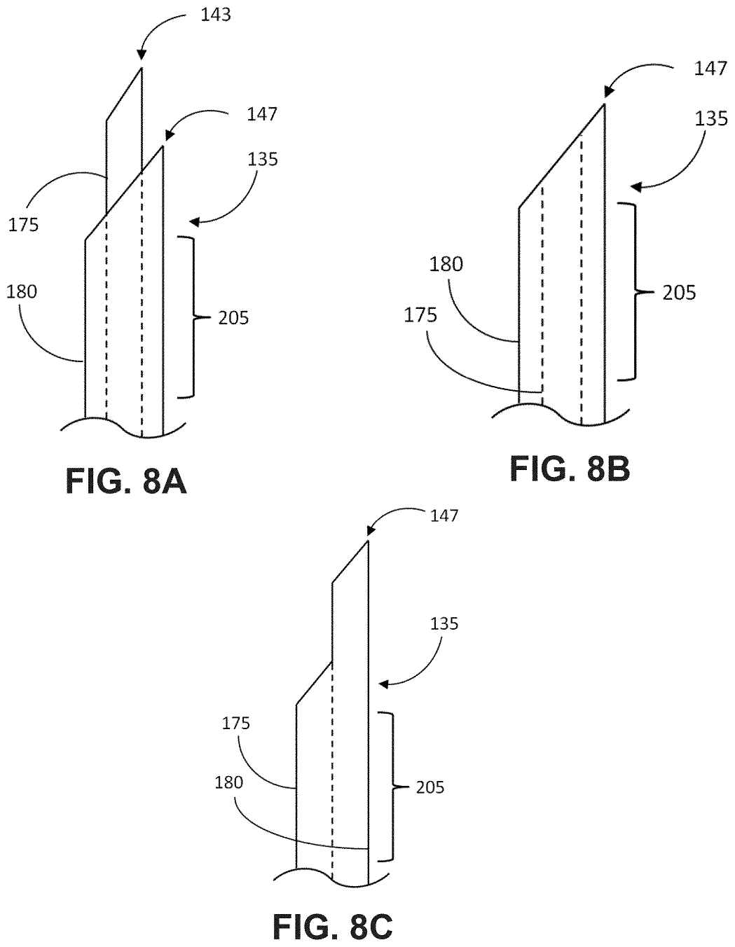

An end portion of the first extension 175 extends out axially from the main body 136 beyond an axial end of the second extension 180. Accordingly, in this embodiment, an end portion of the first extension lumen 178 extends out from the second extension lumen 183. As such, in this embodiment, the first aperture 185 is at a different elevation than the second aperture 190. Moreover, the first extension 175 extends axially beyond a port connector 205 portion of the second extension 180. The port connector 205 is explained elsewhere with reference to FIGS. 8A-8C. The first aperture 185 is therefore at a different elevation than the port connector 205 and, in an aspect of the design, extends further into the reservoir 132 than the port connector 205 extends into the reservoir 132.

In addition to the first extension 175 and the second extension 180 extending out from the main body 136 of the reservoir connector assembly 135, an arm 137 can extend out from the main body 136. The arm 137 can extend out from the main body 136 at a location between opposite ends of the main body 136. As shown here, the arm 137 extends out from the main body 136 at a location along the main body 136 between end 157 and end 158. The arm 137 can extend out from the main body 136 at an angle, measured from a central longitudinal axis of the main body 136, less than ninety degrees, less than seventy-five degrees, or less than fifty degrees.

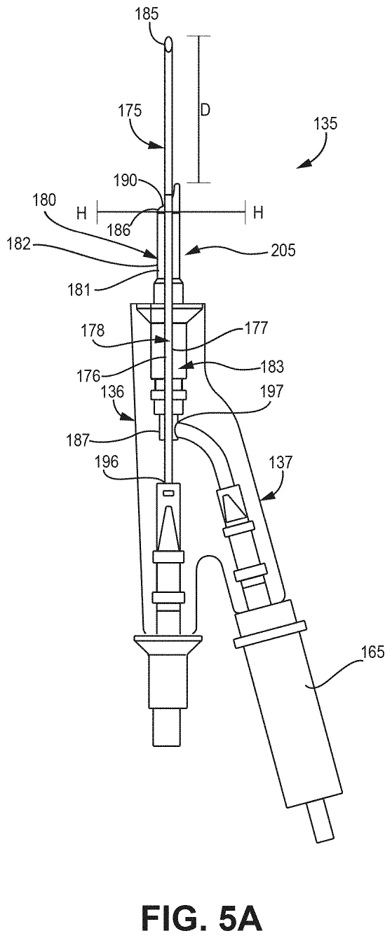

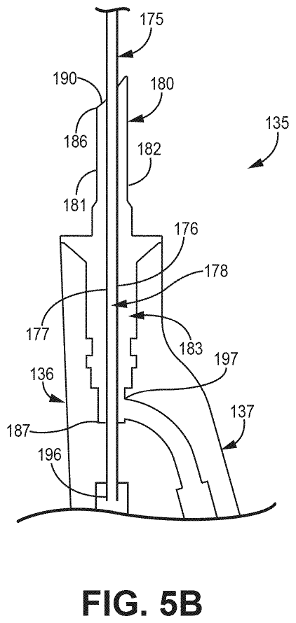

FIGS. 5A and 5B show cross-sectional views, taken along line A-A in FIG. 4, of the reservoir connector assembly 135. FIG. 5B shows a close-up cross-sectional view of a portion of cross-sectional view of the reservoir connector assembly 135 shown in FIG. 5A.

As noted, and shown in FIGS. 5A and 5B, at least a portion of the first extension 175 extends within the second extension 180 such that at least a portion of the first extension lumen 178 extends within the second extension lumen 183 circumventing the first extension lumen 178. In this embodiment, at locations where the first extension 175 extends within the second extension 180, the second extension inner surface 181 bounds both the second extension lumen 183 and the first extension lumen 178. In the illustrated embodiment, the first extension lumen 178 is liquidly isolated from the second extension lumen 183, at least at locations where the first extension lumen 178 extends within the second extension lumen 183. For example, one or both of the first extension inner surface 176 and first extension outer surface 177 can extend along the full length of the second extension lumen 183 to liquid isolate the first and second extension lumens 178, 183. Accordingly, in this configuration, liquid within the first extension lumen 178 can be blocked from passing to the second extension lumen 183 and, likewise, liquid within the second extension lumen 183 can be blocked from passing to the first extension lumen 178.

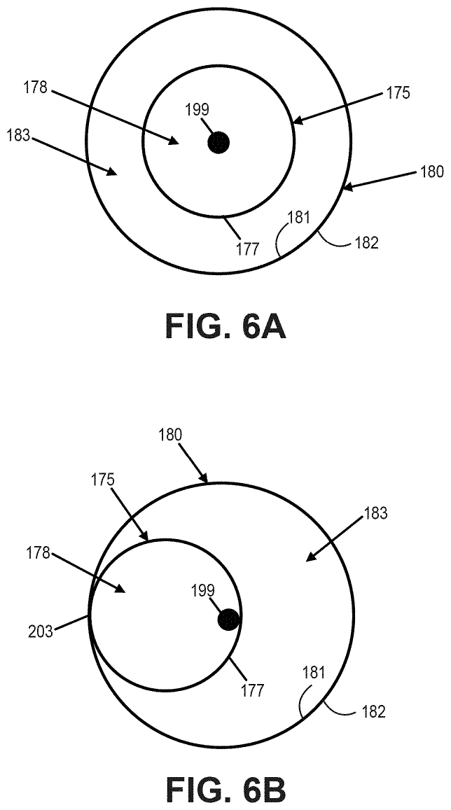

In the illustrated embodiment, the second extension lumen 183 can be defined between the second extension inner surface 181 and the first extension outer surface 177. More specifically, in the embodiment shown, the first extension lumen 178 is concentric with the second extension lumen 183. In this configuration, the first extension outer surface 177 is spaced an equal distance from the second extension inner surface 181 at each longitudinal location where the first extension 175 extends within the second extension 180. Though in other embodiments of the reservoir connector assembly, the first extension lumen 178 can extend within the second extension lumen 183 at a location that is offset from a central longitudinal axis of the second extension lumen 183. This is described further in reference to FIGS. 6A and 6B.

The first extension lumen 178 can extend beyond the second extension lumen 183. For example, as shown in the illustrated embodiment, the first extension lumen 178 can extend beyond a first end 186 of the second extension lumen 183. And, as also shown in the illustrated embodiment, the first extension lumen 178 can extend beyond a second, opposite end 187 of the second extension lumen 183. In particular, in some embodiments, such as that shown here, the first extension lumen 178 can extend through the end 187 of the second extension lumen 183.

To facilitate liquid flow, the reservoir connector assembly 135 can include a port 196 and another port 197. The port 196 can be in liquid communication with one of the first aperture 185 and the second aperture 190. The port 197 can be in liquid communication with the other of the first aperture 185 and the second aperture 190.

In the illustrated embodiment, the port 197 is in liquid communication with the second extension lumen 183. In particular, in this embodiment, the second aperture 190 is a supply aperture and the port 197 is in liquid communication with the second aperture 190. The second aperture 190 can be at the first end 186 of the second extension lumen 183 and the port 197 can be at the second, opposite end 187 of the second extension lumen 183. In the illustrated embodiment, the port 197 can be defined through the second extension inner surface 181 and the second extension outer surface 182 at the second end 187 of the second extension lumen 183.

Accordingly, in the illustrated embodiment, liquid can pass from the reservoir through the second aperture 190 and into the second extension lumen 183. This liquid can travel through the second extension lumen 183 and exit the second extension lumen 183 at the port 197. Thus, the port 197 can be configured to draw liquid supply from the reservoir through the second extension lumen 183. Liquid exiting the port 197 can pass to the drip chamber 165 from which the liquid can then pass to the manifold inlet 155. For instance, the delivery liquid line 145 can be in liquid communication with the port 196 (e.g., via the drip chamber 165) and manifold inlet 155 to convey liquid from the second aperture 190 to the manifold inlet 155.

Also in the illustrated embodiment, port 196 is in liquid communication with the first aperture 185. In particular, in this embodiment, the first aperture 185 is a return aperture and the port 196 is in liquid communication with the first aperture 185. The first aperture 185 can be at a first end of the first extension lumen 178 and the port 196 can be at a second, opposite end of the first extension lumen 178. The first extension lumen 178 can, as in the illustrated embodiment, have a uniform cross-sectional area along its length from the first aperture 185 to the port 196.

Accordingly, in the illustrated embodiment, liquid can pass from the manifold outlet 160 through the port 196 and into the first extension lumen 178. This liquid can travel through the first extension lumen 178 and exit the first extension lumen 178 at the first aperture 185 where it is returned into the reservoir 132. Thus, the port 196 can be configured to return liquid to the reservoir through the first extension lumen 178. For instance, the return liquid line 150 can be in liquid communication with the manifold outlet 160 and the port 196 to convey liquid from the manifold outlet 160 to the first aperture 185.

Reservoir 132, such as commonly available saline bags, typically contains a volume of air. In an aspect of the reservoir connector assembly 135, it may be advantageous for the connector assembly 135 to not receive air from the reservoir 132. Moreover, to the extent air bubbles are present in first extension 175 or the second extension 180, Applicant submits that it would be undesirable for such air bubbles to be output from the connector assembly 135 into the reservoir 132 via the first aperture 185 or the second aperture 190 and then re-enter the connector assembly 135 via the other of the first aperture 185 or the second aperture 190. Air bubbles in the connector assembly 135 may enter into and travel through the delivery liquid line 145 introduce bubbles into the liquid cooling passages of the medical device 115 and the second medical device 125. Such bubbles may even remain in the liquid cooling passages of the medical device 115 and the second medical device 125. Since the bubbles do not provide as efficient of a heat transfer (cooling) as the liquid, bubbles in the liquid cooling passages of the medical device 115 and the second medical device 125 may lead to inconsistent cooling, or even no cooling, of one or more medical devices 115, 125, which may impact the ablation size and performance and/or result in the medical devices malfunctioning or becoming damaged.

In an aspect of the reservoir connector assembly 135, the first aperture 185 can be seen protruding a distance D out further from the main body 136 than the second aperture 190. Similarly, the first aperture 185 can be seen protruding at least a distance D out further from the port connector 205. In such embodiments, this may allow the first aperture 185 to protrude further into a reservoir (e.g. reservoir 132) than second aperture 190. In optional embodiments, the distance D may be greater than 0.5 cm, greater than 1 cm, greater than 2 cm, or about 3 cm. However other distances have been contemplated. Furthermore, the illustrated embodiments may allow first aperture 185 to be elevated higher in the reservoir than second aperture 190. At most, the first aperture 185 may be elevated up to the top of the height of the reservoir. Additionally, the first aperture 185 may be in liquid communication with the manifold outlet 160 and the second aperture 190 may be in liquid communication with the manifold inlet 155. In such an embodiment, the first aperture 185 functions as an outlet aperture and the second aperture 190 functions as an inlet aperture. To the extent air bubbles are output from the first aperture 185, such air bubbles would be introduced into the reservoir 132 and may rise upward towards the top of the reservoir 132. Similarly, liquid from the reservoir 132 may be input into the second aperture 190 at a lower elevation. The distance D between the first aperture 185 and the second aperture 190 may provide enough separation such that any air bubbles released from first aperture 185 are not reintroduced into the connector assembly 135 via the second aperture 190. Moreover, the distance D between the first aperture 185 and the second aperture 190 may provide enough separation such that any liquid output from one of the apertures (185, 190) is not almost immediately reintroduced into the connector assembly 135 via the other of the apertures (185, 190). If liquid output one aperture was allowed to flow directly into the other aperture, the cooling provided by the liquid might be compromised. That is, liquid output one aperture is warmed during its traversal through a microwave needle ablation probe. To the extent such liquid is almost immediately reintroduced into the connector assembly 135, it is not given a chance to mix with and be cooled by other liquid in the reservoir 132, and therefore such relatively warmer liquid will provide less cooling of the microwave ablation probe as it traverses such probe again. In addition, since the second aperture 190 sits a distance D below the first aperture 185, the water pressure of the fluid in the reservoir 132 is greater at the second aperture 190 than at the first aperture 185. The additional water pressure assists with the fluid flow into the reservoir connector assembly 135 if the second aperture 190 is functioning as the inlet. Similarly, the relatively lower water pressure at the first aperture 185 provides less resistance to fluid flow out of the reservoir connector assembly 135 if the first aperture functions as the outlet.

In another embodiment of the reservoir connector assembly, the flow direction of the recirculating liquid flow can be reversed. In such an embodiment of the reservoir connector assembly, the supply would be in liquid communication with the first aperture and first extension lumen while the return would be in liquid communication with the second aperture and the second extension lumen. In this embodiment, liquid could enter the first extension lumen from the reservoir and exit through the outlet at the first extension lumen to the manifold inlet. Also in this embodiment, liquid could exit the manifold outlet, enter the second extension lumen, and pass through the second extension lumen into the reservoir.

FIGS. 6A and 6B are cross-sectional views, taken along a plane perpendicular to line A-A in FIG. 4 at about the height of the extensions shown at line H-H in FIG. 5A. FIGS. 6A and 6B show example embodiments of the relative positioning of the first extension 175 and the second extension 180 of the reservoir connector assembly 135 described herein. As noted, the first extension 175 can define the first extension lumen 178 and the second extension 180 can define the second extension lumen 183. As illustrated in FIGS. 6A-6B, the first extension lumen 178 can extend within the second extension lumen 183 at a variety of locations, including a location that is concentric with the second extension lumen 183 and a location that is offset from a central longitudinal axis 199 of the second extension lumen 183.

FIG. 6A illustrates an embodiment of the relative positioning of the first extension 175 and the second extension 180 that can be present in the embodiment of the reservoir connector assembly 135 described herein. In the embodiment shown in FIG. 6A, the second extension lumen 183 is defined between the second extension inner surface 181 and the first extension outer surface 177. More specifically, in the embodiment shown, the first extension lumen 178 is concentric with the second extension lumen 183. Here, the central longitudinal axis 199 of the second extension lumen 183, and the second extension 180, is also the central longitudinal axis of the first extension lumen 178, and the first extension 175. In this configuration, the first extension outer surface 177 is spaced an equal distance from the second extension inner surface 181 at each longitudinal location where the first extension 175 extends within the second extension 180.

FIG. 6B illustrates another embodiment of the relative positioning of the first extension 175 and the second extension 180 that can be present in the embodiment of the reservoir connector assembly 135 described herein. In the embodiment shown in FIG. 6B, the first extension lumen 178, and first extension 175, extends within the second extension lumen 183 at a location that is offset from the central longitudinal axis 199 of the second extension lumen 183, and the second extension 180. In this configuration, one side of the first extension outer surface 177 can be closer to the second extension inner surface 181 than another side of the first extension outer surface 177 at one or more longitudinal locations where the first extension 175 extends within the second extension 180. Such one or more locations may be at all longitudinal locations, and it may be at least at the longitudinal location of the second aperture 190, and such an offset location may extend the longitudinal distance D that first extension 175 extends beyond second extension 180. In particular, the illustrated embodiment where the first extension lumen 178 is offset from the central longitudinal axis 199 of the second extension lumen 183, a portion of the second extension inner surface 181 and a portion of the first extension outer surface 177 can be a common surface 203. In this embodiment, a portion the first extension 175 can be in contact with a portion of the second extension inner surface 181 such that liquid within the second extension lumen 183 does not pass between the second extension inner surface 181 and the first extension 175 at the location (common surface 203) where the location where the portion the first extension 175 is in contact with the portion of the second extension inner surface 181.

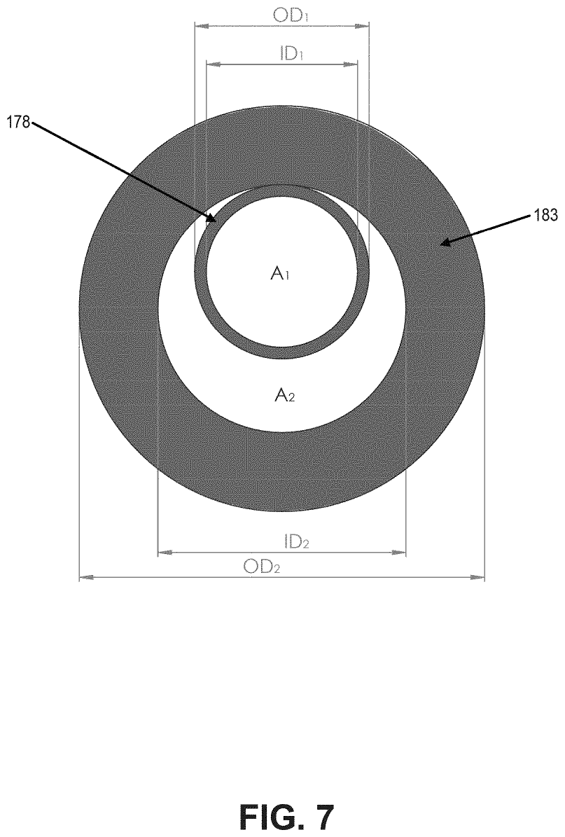

FIG. 7 is a cross-sectional view, taken along a plane perpendicular to line A-A in FIG. 4 at about the height of the extensions shown at line H-H in FIG. 5A. FIG. 7 illustrates one embodiment of the relative cross-sectional areas of the extensions in the embodiment of the reservoir connector assembly shown in FIG. 1. That is, FIG. 7 may represent the cross-section of the first extension 175 and the second extension 180 at line H-H that shows the relative sizes and locations of the first extension lumen 178 and the second extension lumen 183. Such cross-section is intended to show the smallest cross-sectional areas of such first extension lumen 178 and the second extension lumen 183. Instead of being a cross-section at line H-H, though, FIG. 7 may represent a two-dimensional projection of the first extension 175 and the second extension 180 (e.g., a view, looking downward from line A-A in FIG. 4 at first aperture 185 and second aperture 190) such that the first extension lumen 178 and the second extension lumen 183 shown in FIG. 7 represent the relative sizes and locations of the first aperture 185 and the second aperture 190, respectively. Such projection is also intended to show the smallest cross-sectional areas of the first extension lumen 178 and the second extension lumen 183.

In the embodiment shown in FIG. 7, the first extension lumen 178 may compromise a first inner diameter (ID₁) outlining the first extension inner surface and a first outer diameter (OD₁) outlining the first extension outer surface and the second extension lumen 183 may comprise a second inner diameter (ID₂) outlining the second extension inner surface and a second outer diameter (OD₂) outlining the second extension outer surface. In particular embodiments, ID₁ is between 1.45 mm and 2 mm, such as 1.83 mm; OD₁ is between 1.8 mm and 2.8 mm, such as 2.11 mm; ID₂ is between 2.5 mm and 3.2 mm, such as 3 mm; and OD₂ is between 4.5 mm and 5.5 mm, such as 4.9 mm. In optional embodiments, ID₁ is less than OD₁ and ID₂ is less than OD₂. Additionally, OD₁ may be less than ID₂, for example, when the first extension lumen 178 is fully and/or partially enclosed within the second extension lumen 183. In the embodiment illustrated in FIG. 7, the cross-sectional area of the first aperture (A₁) may be defined as the area inside ID₁ and the cross-sectional area of the second aperture may be defined as the area inside ID₂ and outside OD₁ (area inside ID₂ minus the area outside OD₁). That is, in some aspects of the design, A₁ may represent the cross-sectional area of first aperture 185 and A₂ may represent the cross-sectional area of second aperture 190. A₁ may also represent the cross-sectional area inside the first extension 175 and A₂ may represent the cross-sectional area inside the second extension 180 minus the cross-sectional area within the outside of the first extension 175, again where such cross-sectional areas are at their smallest within the first extension lumen 178 and the second extension lumen 183.

In another aspect of the relative areas, A₁ may correspond to an outlet cross-sectional area (e.g., outlet aperture area or area of aperture within an extension functioning as an outlet) and A₂ may correspond to an inlet cross-sectional area (e.g., inlet aperture area or area of aperture within an extension functioning as an inlet). That is, in such aspect of the invention, first extension 175 and first aperture 185 may be functioning as an outlet having a cross-sectional aperture area of A₁ and second extension 180 and second aperture 190 may be functioning as an inlet having a cross-sectional aperture area of A₂. In such aspect of the invention, the ratio of A₁ to A₂ (A₁/A₂) corresponds to a ratio of outlet area to inlet area.

In optional embodiments where A₁ corresponds to an outlet cross-sectional area and A₂ corresponds to an inlet cross-sectional, A₁ is larger than A₂ such that the cross-sectional areas of the aperture connected to the outlet is smaller than the cross-sectional areas of the aperture connected to the inlet. In particular embodiments, A₁ is between 2 mm² and 3.5 mm², such as 2.63 mm² and A₂ is between 3 mm² and 5 mm² such as 3.57 mm². Additionally or alternatively, the ratio of A₁ to A₂ (A₁/A₂) may be between 0.65 and 0.85, and optionally around 0.74. Stated differently, A₁ may be between 65% and 85% of the size of A₂, such as around 74%, or A₂ may be between 15% and 35% larger than A₁ and, such as around 26% larger.

In such embodiments where A₁ corresponds to an outlet cross-sectional area and A₂ corresponds to an inlet cross-sectional, having A₁ be smaller than A₂ may help balance the pressure in lines (e.g. the delivery liquid line 145 and the return liquid line 150 with respect to FIG. 1) which may help an attached pump maintain a stable and consistent flow rate as well as provide a higher efficiency to the system, such as an attached pump working at or closer to an optimal efficiency or the attached pump less susceptible to stalling and/or failure. Such embodiments may help maintain a stable and consistent temperature in the one or more fluidly connected medical devices (e.g. medical device 115). Since the reservoir connector assembly 135 uses a single port connector 205 to connect to a single connection port 172 of a reservoir, the cross-sectional areas A₁ and A₂ are fairly small. With such relatively small cross-sectional areas, A₁ and A₂, it is relatively difficult to achieve high flow rates through the reservoir connector assembly 135. By using the relatively larger cross-sectional area, A₂, as the inlet, the larger helps provide a larger flow rate in.

The first aperture 185 can be seen protruding a distance D out further from the main body 136 than the second aperture 190. Similarly, the first aperture 185 can be seen protruding at least a distance D out further from the port connector 205.

It should be understood that other ratios, percentages, and sizes, such as smaller and larger than the disclosed sizes for ID₁, OD₁, ID₂, OD₂, A₁, and A₂, are also within the scope of this aspect of the design. For instance, Alternatively, A₁ may be less than A₂ or the same area as A₂. Furthermore, even though the illustrated cross section of the first extension lumen 178 and the second extension lumen 183 is circular, the cross-sectional areas may comprise alternative shapes, such as ovals, rectangles, pentagons, or various regular or irregular polygons.

Thus, the reservoir connector assembly 135 disclosed herein can be configured to facilitate recirculating liquid flow, such as between the reservoir and the manifold. Embodiments of the reservoir connector assembly 135 disclosed herein can do so with a connection to a single connection port of a reservoir. As such, these embodiments do not require connections at two separate connection ports of a reservoir and can thereby facilitate easier system setup and reduce the number of component parts resulting in a more user friendly and cost efficient connector assembly. At the same time, embodiments of the reservoir connector assembly 135 disclosed herein can provide both an efficient liquid delivery pathway and an efficient liquid return pathway within the reservoir connector assembly.

It is common for the connection port 172 of reservoir 132 to be sealed closed, such that the contents in the main volume 170 do not leak and/or spill out before reservoir connector assembly 135 is initially connected. In an aspect of the connector assembly 135, the connector assembly 135 may comprise a tip capable of penetrating the seal on connection port 172 (forming a single orifice). In particular embodiments, at least one of the first extension 175 and the second extension 180 may comprise structure for penetrating the seal on connection port 172. Such structure may include sufficient rigidity to penetrate the seal. Such structure may include one or more sharp tips or edges that assist in penetrating the seal. Moreover, in an aspect of the connector assembly 135, the connector assembly 135 includes a port connector that provides a connection to a single connection port of a reservoir. For reservoir 132 with a connection port 172 that is designed to receive a port connector in the connection port 172, aspects of the port connector of the current disclosure include a tube that is inserted in the connection port 172 and sealed to the connection port 172 to prevent leakages or spills.

Additionally or alternatively, at least one of the first extension 175 and the second extension 180 may comprise a spike, such as first spike 143 and second spike 147 as shown in FIG. 8A. FIG. 8A provides exemplary cross sectional views, taken along line A-A in FIG. 4 of the reservoir connector assembly 135 of the first extension 175 and the second extension 180. In the embodiment of FIG. 8A, the first extension 175 is located within the second extension 180 and the first extension 175 extends above the second extension 180. One or more spikes may be configured to penetrate the seal on connection port 172. In such embodiments, the first spike 143 may be configured to be received by connection port 172 as well as penetrate the seal on connection port 172 in order to allow the first extension 175 to extend into the main volume 170 of reservoir 132. Additionally or alternatively, the second spike 147 may be configured to be received by connection port 172 as well as penetrate the seal on connection port 172 in order to allow the second extension 180 to extend into the main volume 170 of reservoir 132. When connector assembly 135 is inserted into connection port 172, the first spike 143 may penetrate connector port 172 followed by the second spike 147 as connector assembly 135 is inserted further into reservoir 132.

FIG. 8A also shows the approximate location of the port connector 205 (see also port connector 205 in FIG. 5A). Port connector 205 provides a connection to a single connection port of a reservoir. Port connector 205 in FIGS. 5A and 8A has a cylindrical outer surface that seals against the inner surface of a connection port 172 of a reservoir 132. Port connector 205 in FIGS. 5A and 8A is formed from a lower portion of second extension 180. Thus, the outer surface of section extension 180 provides the seal against the inner surface of a connection port 172 at a location of section extension 180 somewhere within the height of port connector 205. Accordingly, in the design shown in FIG. 8A, port connector 205 has two spikes, first spike 143 and second spike 147, to assist with insertion in a connection port 172. When connector assembly 135 is inserted into connection port 172, the first spike 143 may penetrate connection port 172 followed by the second spike 147, and, as connector assembly 135 is inserted further into reservoir 132, port connector 205 is inserted in and seals the connection port 172.

In some aspects of the design of the port connector 205, port connector 205 only has one spike. For instance, in the embodiment shown in FIG. 8A, one of first spike 143 and second spike 147 could be eliminated. In such case, either first spike 143 or second spike 147 could pierce the seal sufficiently and permit port connector 205 to be able to be inserted far enough into the connection port 172 to seal off the reservoir 132.

FIG. 8B shows another such example of using a single spike. FIG. 8B is similar to the design in FIG. 8A, except that first extension 175 does not extend beyond second extension 180. That is the distance D in FIG. 5A is zero in the aspect of the connector assembly 135 design shown in FIG. 8B. This aspect of the design of the connector assembly 135 is the same as FIG. 8A, but the terminal end of first extension 175, adjacent to spike 147, does not have a spike. Accordingly, in the design shown in FIG. 8B, port connector 205 has one spike, spike 147, to assist with insertion in a connection port 172 and pierce the seal of the reservoir 132. When connector assembly 135 of FIG. 8B is inserted into connection port 172, the second spike 147 may penetrate connector port 172, and, as connector assembly 135 is inserted further into reservoir 132, port connector 205 is inserted in and seals the connection port 172.

FIG. 8C shows another such example of using a single spike. FIG. 8C is similar to the design in FIG. 8A, except that first extension 175 is adjacent to, instead of inside of, second extension 180. This aspect of the design of the connector assembly 135 is similar to that of FIG. 8A, but the terminal end of first extension 175 does not have a spike 147.

In addition, instead of port connector 205 being formed from a lower portion of section extension 180, port connector 205 in FIG. 8C is formed from a combination of the lower portions of section extension 180 along with first extension 175. Accordingly, in the design shown in FIG. 8C, port connector 205 has one spike, spike 147, to assist with insertion in a connection port 172 and pierce the seal of the reservoir 132. When connector assembly 135 of FIG. 8C is inserted into connection port 172, the second spike 147 may penetrate connector port 172, and, as connector assembly 135 is inserted further into reservoir 132, port connector 205 is inserted in and seals the connection port 172.

Also within the scope of the present disclosure are a number of methods relating to liquid connections, liquid flow, and manufacture of a reservoir connector assembly.

One method embodiment includes a method of making a reservoir connector assembly. This method embodiment can include a step of forming a first extension and a second extension. The first extension can be formed to include a first extension inner surface, a first extension outer surface, and a first extension lumen that is defined by the first extension inner surface. The second extension can be formed to include a second extension inner surface, a second extension outer surface, and a second extension lumen that is defined by the second extension inner surface. When so formed, at least a portion of the first extension lumen can extend within the second extension lumen. Also at this step, the first extension lumen can be formed to include a first aperture at a first tip that is configured to pierce a connection port at a reservoir and the second extension lumen can be formed to include a second aperture at a second tip, at a different elevation than the first tip, configured to pierce the same connection port at the reservoir. This method can include a further step of forming an outlet in liquid communication with one of the first aperture and the second aperture and an inlet in liquid communication with the other of the first aperture and the second aperture. In a further embodiment of this method, the first extension lumen can be formed so as to be liquidly isolated from the second extension lumen. In yet further embodiments of this method, the reservoir connector assembly can be formed to include any one or more other features disclosed herein.

Another method embodiment includes a method of recirculating liquid within a reservoir connector assembly. This method embodiment can include any one or more steps relating to liquid entering the reservoir connector assembly described herein from a reservoir, passing through the reservoir connector assembly described herein to a manifold inlet, re-entering the reservoir connector assembly described herein from a manifold outlet, and passing through the reservoir connector assembly described herein back to, and through, the same connection port at the reservoir through which the liquid initially entered the reservoir connector assembly.

Various non-limiting exemplary embodiments have been described. It will be appreciated that suitable alternatives are possible without departing from the scope of the examples described herein. These and other examples are within the scope of the following claims.

1. A reservoir connector assembly for connecting to a single connection port of a fluid reservoir, comprising:
a first extension configured to extend into the single connection port and including a first extension inner surface, a first extension outer surface, and a first extension lumen that is defined by the first extension inner surface, the first extension lumen including a first aperture that is configured to be positioned within the reservoir;
a second extension configured to extend into the single connection port and including a second extension inner surface, a second extension outer surface, and a second extension lumen that is defined by the second extension inner surface, at least a portion of the first extension lumen extending within the second extension lumen, the second extension lumen including a second aperture that is configured to be positioned within the reservoir;
an outlet in liquid communication with one of the first aperture and the second aperture; and
an inlet in liquid communication with the other of the first aperture and the second aperture.

2. The reservoir connector assembly of claim 1, wherein the second extension outer surface entirely surrounds the first extension lumen.

3. The reservoir connector assembly of claim 1, wherein a portion of the second extension inner surface and a portion of the first extension outer surface are a common surface.

4. The reservoir connector assembly of claim 1, wherein the first extension lumen is concentric with the second extension lumen.

5. The reservoir connector assembly of claim 1, wherein the reservoir connector assembly includes a port connector having an outer surface that is configured to be received in and seal against the single connection port of the reservoir, the outer surface of the port connector being the outer surface of the second extension.

6. The reservoir connector assembly of claim 1, wherein the first aperture is at a different elevation than the second aperture.

7. The reservoir connector assembly of claim 1, wherein the first aperture is an output aperture and the outlet is in liquid communication with the first aperture, and wherein the first aperture is at a first end of the first extension lumen and the outlet is at a second end of the first extension lumen, the first end being opposite the second end.

8. The reservoir connector assembly of claim 1, further comprising a drip chamber located downstream of the outlet.

9. The reservoir connector assembly of claim 1, wherein the second aperture is an inlet aperture and the inlet is in liquid communication with the second aperture, and wherein the second aperture is at a first end of the second extension lumen and the inlet is at the second extension inner surface and the second extension outer surface at a second end of the second extension lumen, the first end being opposite the second end.

10. A reservoir connector assembly for connecting to a connection port of a fluid reservoir, comprising:
a first extension configured to extend into the connection port and into the reservoir, the first extension including a first extension inner surface, a first extension outer surface, and a first extension lumen that is defined by the first extension inner surface, the first extension lumen including a first aperture that is configured to be positioned within the reservoir;
a second extension configured to extend into the connection port, the second extension including a second extension inner surface, a second extension outer surface, and a second extension lumen that is defined by the second extension inner surface, the second extension lumen including a second aperture that is configured to be positioned within the reservoir, the first extension lumen extending further into the reservoir than the second extension lumen, the first aperture positioned further into the reservoir than the second aperture;
an outlet in liquid communication with the second aperture; and
an inlet in liquid communication with the first aperture.

11. The reservoir connector assembly of claim 10, wherein at least a portion of the first extension lumen extends within the second extension lumen.

12. The reservoir connector assembly of claim 10, wherein a portion of the second extension inner surface and a portion of the first extension outer surface are a common surface.

13. The reservoir connector assembly of claim 10, wherein the first extension lumen is concentric with the second extension lumen.

14. The reservoir connector assembly of claim 10, wherein the reservoir connector assembly includes a port connector having an outer surface that is configured to be received in and seal against the connection port of the reservoir, the outer surface of the port connector being the outer surface of the second extension.

15. The reservoir connector assembly of claim 10, wherein the first extension terminates in a spike for insertion in and penetrating a seal in the connection port, and the second extension terminates in a spike for insertion in and penetrating the seal in the connection port.

16. The reservoir connector assembly of claim 10, wherein the first aperture is an output aperture and the outlet is in liquid communication with the first aperture, and wherein the first aperture is at a first end of the first extension lumen and the outlet is at a second end of the first extension lumen, the first end being opposite the second end.

17. The reservoir connector assembly of claim 10, wherein the second aperture is an inlet aperture and the inlet is in liquid communication with the second aperture, and wherein the second aperture is at a first end of the second extension lumen and the inlet is at the second extension inner surface and the second extension outer surface at a second end of the second extension lumen, the first end being opposite the second end.

18. The reservoir connector assembly of claim 10, wherein the cross-sectional area of the second aperture is larger than the cross-sectional area of the first aperture.

19. A method of connecting a reservoir connecting assembly to a single connection port of a fluid reservoir, the reservoir connecting assembly comprising:
a first extension including a first extension inner surface, a first extension outer surface, and a first extension lumen that is defined by the first extension inner surface, the first extension lumen including a first aperture that is configured to be positioned within the reservoir;
a second extension including a second extension inner surface, a second extension outer surface, and a second extension lumen that is defined by the second extension inner surface, at least a portion of the first extension lumen extending within the second extension lumen, the second extension lumen including a second aperture that is configured to be positioned within the reservoir;
connecting the first extension into the single connection port; and
connecting the second extension into the single connection port.

20. A method according to claim 19, further comprising:
connecting a delivery liquid line to an outlet of the reservoir connecting assembly, and
connecting a return liquid line to an inlet of the reservoir connecting assembly.
